(19) United States
(12) Patent Application Publication
(10) Pub. No.: US 2012/0172833 A1
(43) Pub. Date: Jul. 5, 2012

(54) SYSTEM AND METHOD FOR CONTROLLING DRUG DELIVERY

(76) Inventor: Yehuda ZISAPEL, Tel Aviv (IL)

(21) Appl. No.: 13/343,772

(22) Filed: Jan. 5, 2012

Related U.S. Application Data
(60) Provisional application No. 61/429,985, filed on Jan. 5, 2011.

(51) Int. Cl. A61M 5/168

(52) U.S. Cl. 604/503; 604/66

(57) ABSTRACT

The present invention relates to a system for delivering a drug to a patient, which comprises at least one electro-physical sensor, a control unit and a drug-delivery device adapted to contain at least one drug to be delivered to such patient. The sensor and control unit are capable of determining a physiological state in a patient wearing the sensor, and the control unit is capable of issuing instructions to the drug-delivery device so as to deliver the drug to the patient at a flow-rate which is a function of the physiological state determined.

### CROSS-REFERENCE TO RELATED APPLICATIONS

This application claims the benefit of U.S. Provisional Application No. 61/429,985, filed Jan. 5, 2011, the contents of which is hereby incorporated by reference herein in its entirety.

### FIELD OF THE INVENTION

The present invention is in the field of drug delivery. Specifically, the invention is in the field of delivering drugs such as hormones to patients, while implementing a specific controlled, drug-delivery regimen so that the level of hormone in the patient will mimic either the natural hormone levels in the body according to its biological clock time, or synchronized with the sleep slow-wave period.

### BACKGROUND

Hormones are chemicals which are released by a cell or a gland in one part of the body which sends out messages that affect cells in other parts of the organism. Only a small amount of hormone is required to alter cell metabolism. In essence, they are chemical messengers that transport a signal from one cell to another. They are secreted naturally by humans and other animals either directly or indirectly (via a duct) into the bloodstream. There are numerous medical indications where hormones need to be given to a patient either because the naturally released quantities of that hormone are insufficient, or because a specific medical outcome requires the attainment of an enhanced hormonal level beyond the normal or natural level. Examples of such indications include growth hormone (GH) supplementation for patients (typically children) suffering from growth hormone deficiency (GHD), GH supplementation for improving the growth and regulation of muscle and metabolism in aging patients, and Gonadotropin-releasing hormone (GnRH) for women undergoing fertility treatment.

Growth hormone (GH) is a protein-based peptide hormone. It stimulates growth, cell division and regeneration in humans and other animals. Growth hormone is a 191-amino acid, single-chain polypeptide that is synthesized, stored, and secreted by the somatotroph cells within the lateral wings of the anterior pituitary gland. Growth Hormone Deficiency (GHD) is a medical condition in which the body does not produce enough growth hormone (GH). Growth hormone is used as prescription drug in medicine to treat children's growth disorders and adult growth hormone deficiency and other indications.

Gonadotropin-releasing hormone (GnRH) is a tropic peptide hormone responsible for the release of follicle-stimulating hormone (FSH) and luteinizing hormone (LH) from the anterior pituitary. GnRH is naturally synthesized and released from neurons within the hypothalamus. GnRH is also available as gonadorelin hydrochloride for injectable use, typically via an infusion pump system to induce ovulation in patients.

The natural hormonal pattern of hormone release is typically pulsatile and it is critical that the pulsatility be synchronized to the biological clock for obtaining the proper physiological response. For example, GH is synthesized and secreted from the anterior pituitary gland in a pulsatile manner throughout the day/night cycle; surges of secretion typically occur at 3- to 5-hour intervals. The plasma concentration of GH during these peaks may range from 5 to 45 ng/mL. The largest and most predictable of these GH peaks occurs about an hour after onset of sleep. Numerous physiological stimulators affect GH release, including entry into a deep sleep stage. Research in this field, such as that by Van E. Cauter et. al., “Reciprocal interactions between the GH axis and sleep” (Growth Horm. IGF Res. 14 Suppl A: S10-7, June 2004), which is incorporated herein by reference, describes the relationship between sleep states and the production of GH, and reports that growth hormone (GH) is preferentially secreted during deep, slow-wave sleep (SWS). With aging, SWS is reduced and GH secretion is reduced as well, which may lead to a decline in muscle mass in the elderly. Another example is GnRH, by which pulsatile secretion enhances fertility whereas continuous secretion leads to down regulation of the gonadal axis. Continuous supply of GnRH is thus used for treatment of prostate cancer in men and lowering sexual drive in sex offenders.

Accordingly, many studies have been performed in which the delivery of hormones has been performed according to a pulsatile regimen so as to mimic or somewhat mimic the natural release pattern. This is also the reason that GH injections are typically given just before sleep. However, such studies do not involve monitoring the actual physiological cycle in order to provide an optimal hormone delivery regimen in terms of the cycle.

### SUMMARY OF THE INVENTION

In one aspect, the present invention relates to a system for delivering a drug to a patient, wherein the system includes at least one electro-physical sensor, a control unit, and a drug-delivery device adapted to contain at least one drug to be delivered to the patient. The sensor and the control unit are capable of determining a physiological state in a patient wearing the sensor. The control unit is capable of issuing instructions to the drug-delivery device to deliver the drug to the patient at a flow-rate which is a function of the physiological state determined.

In another aspect, the present invention relates to a method of delivering a drug to a patient in need of such drug, the method comprising:

(a) determining a physiological state in such patient by means of an electro-physical sensor in communication with a control unit;

(b) using said control unit to issue instructions to a drug delivery device; and

(c) administering said drug to said patient by means of said drug delivery device at a flow-rate which is a function of the physiological state determined.

### BRIEF DESCRIPTION OF THE DRAWINGS

FIG. 1 provides a schematic block diagram of the system of the present invention.

FIG. 2 is an exemplary illustration of sleep states during sleeping.

FIG. 3 shows profiles of 24 hour serum growth hormone (GH) in human female (top panel) and male (bottom panel) subjects. The profile graphs are reproduced from FIG. 1 in a study by Muller et al. 1999, Physiological Reviews, 79(2), p. 511-607, which is incorporated herein by reference.

FIG. 4 shows profiles of 24 hour serum growth hormone concentration in 29 human children. The profile graph is reproduced from FIG. 2 in a study by Hindmarsh et al. 2005, Arch Dis Child, 90, p. 1144-7, which is incorporated herein by reference.

### DETAILED DESCRIPTION

The current invention seeks to improve the extent to which drug delivery can more accurately achieve the optimal pattern required by taking into account data indicative of the actual physiological/biological time clock cycle of the patient. To that end, a relevant physiological parameter is monitored, and this parameter is used in the algorithm determining the flow rate to be used by the system in delivering the drug, which in a preferred embodiment is a hormone.

In one embodiment, the invention comprises a system for delivering hormones having at least one electro-physical sensor, a control unit and a drug-delivery device containing at least one hormone to be delivered. The sensor and the control unit are capable of determining a physiological state in a patient wearing the sensor. The control unit is capable of issuing instructions to the drug-delivery device to deliver hormone to the patient at a flow-rate, which is a function of the physiological state determined. For example, in one embodiment, the physiological state is a sleep-state. In one embodiment the drug-delivery device includes a pump.

In one embodiment, the hormone flow-rate delivered by the invention follows a preset profile pattern of hormone release. For example, the preset pattern may be the natural hormone release profile of an average adult male if the patient is an adult male, the natural hormone release profile of an average adult female if the patient is an adult female, or the natural hormone release profile of an average child if the patient is a child.

In one embodiment, the drug-delivery device delivers missing quantities of hormone at appropriate times according to the patient's body biological clock. For example, the patient may be diagnosed as hormone deficient and prescribed hormone supplementation therapy. In one embodiment, the control unit can include a polysomnography controller and the sensor can include an EEG-type sensor. In another embodiment, the control unit and the sensor include an actigraph and/or heart rate sensor.

In one embodiment, the control unit is a 24 hour clock synchronized with the patient's body biological clock and sleep time and capable of issuing instructions to the drug delivery device for delivering appropriate amounts of hormone at appropriate times in order to mimic naturally released hormone amounts over time.

In one embodiment, the control unit is activated with sleep time and issues instructions when slow-wave sleep is expected in the natural shape and level of the release of the GH.

In one embodiment, the hormone quantities level, time, and shape can be programmed in the control unit, which is personalized to the patient, in order to dictate the level of hormone released by the pump to the body.

In one embodiment, the drug-delivery device is a patch-pump adhered to the patient's body. In another embodiment, the sensor is wirelessly connected to the control unit. In an embodiment, the sensor comprises the control unit.

In one embodiment, the drug-delivery device is wirelessly connected to the control unit. In an embodiment, the drug-delivery device comprises the control unit.

In one embodiment, the hormone to be delivered is at least one of growth hormone and gonadotropin-releasing hormone. In another embodiment, the hormone to be delivered is any hormone released in the body in a pattern and/or at the levels that are synchronized with the body biological clock.

In one aspect, the invention provides a method of treatment for treating growth hormone deficiency using the system of the invention. In one embodiment, the method involves determining a physiological state in a patient and delivering growth hormone to the patient at a flow-rate which is a function of the physiological state determined.

In another aspect, the invention provides a method of treatment for inducing ovulation in patients using the system of the invention. In an embodiment, the method of the invention involves determining a physiological state in a patient and delivering an ovulation inducing hormone to the patient at a flow-rate which is a function of the physiological state determined.

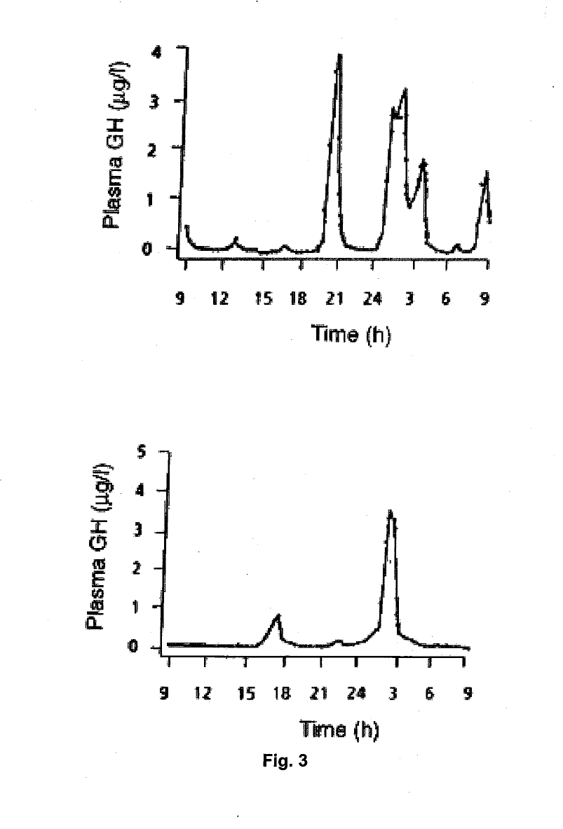

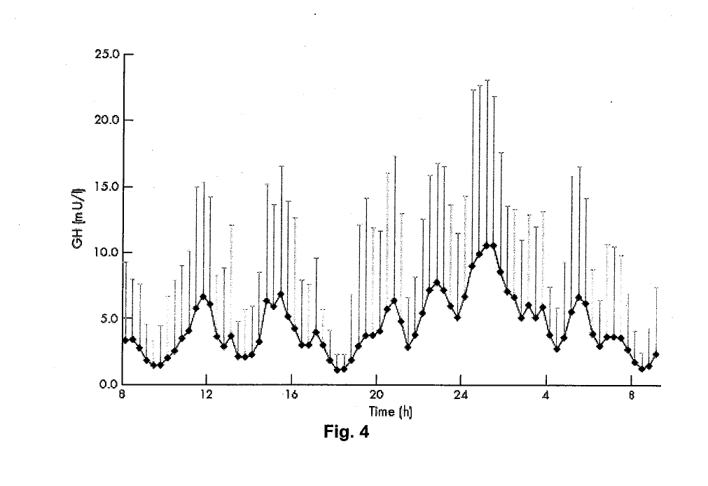

In one embodiment, the invention involves a combination of pulsatory hormone release to mimic the normal gender/age profile of the patient. For example, in the case of delivering GH to children suffering from GHD, the entry into a deep-sleep state and the duration of such state can be used to trigger the start and/or end of a bolus delivery of growth hormone. Alternatively, the start and/or end of the bolus can be triggered by a clock synchronized with the body's biological clock and thereby trigger the hormone delivery pattern to mimic the body's natural pattern of the GH as presented in FIG. 3 or FIG. 4, depending on the age and gender of the patient. Unlike control mechanisms known in the prior art, such as U.S. Pat. No. 6,740,072, which describes a closed-loop control mechanism for delivering a drug in order to compensate for changes in a sensed biological state, the control mechanism of the present invention involves drug delivery which is triggered by a physiological parameter without a compensation element or objective.

A number of means exist to measure relevant physiological parameters indicative of the appropriate timing for bolus or pulsed deliveries. In a preferred embodiment these parameters are derived from electro-physical measurements such as those employing electrical contacts attached to the patient's body, movement sensors, or a time clock synchronized with the body's biological clock (as opposed to chemical or biological sensors), temperature sensors or pH meters. The body's biological clock is described in Prasai et al. “An endocrinologist's guide to the clock,” Journal of Clinical Encdocrinology & Metabolism, 2011, 96(4), 913-22, Golombek and Rosenstein, “Physiology of circadian entrainment,” Physiological Reviews, 2010, 90(3), 1063-102, and Okamura et al. “Mammalian circadian clock system: Molecular mechanisms for pharmaceutical and medical sciences,” Advanced Drug Delivery Reviews, 2010, 62, 876-84, which are each incorporated by reference in their entirety. Electrical sensors can for example be used to determine the EEG signals showing the patient's sleep-state, provide impedance measurements or show skin conductivity. Movement and temperature sensors can also be used in a similar manner so show the sleep-state or other physiological parameters.

Thus, in a preferred embodiment the present invention comprises a sensor as defined above or a time clock, a control unit and a drug-delivery pump where said sensor serves as an input to said control unit such that said control unit determines the flow rate of the drug delivered to the patient in terms of the physiological input provided by said sensor or clock. In a preferred embodiment of said system, at least one and preferably both of the said sensor and said drug-delivery pump have a wireless connection to said control unit; enabling a wire-free and tubing-free connection to the patient for maximum comfort.

Also provided by the present invention is a method of treatment administered using the said system in order to provide an enhanced delivery method for delivering hormones to a human. The GH can be released to the body in the shape, at the levels, and according to the timing of the natural release profile during sleep. This will reduce the overall amount of GH administered to the patient compared to conventional hormone replacement methods and will increase the efficacy of the method. The level of GH preferably will pulse about every 3±2 hours, and the total release quantity during a 24 hour cycle (referred to herein as “T”) will be programmed per patient. The hormone delivery parameters will be programmed based on the normal pharmacological hormone release profile of a healthy subject within the patient's gender and age group. In one embodiment, the release quantity during sleep will be 60%±30% of T and the release quantity during the day will be 40%±30% of T.

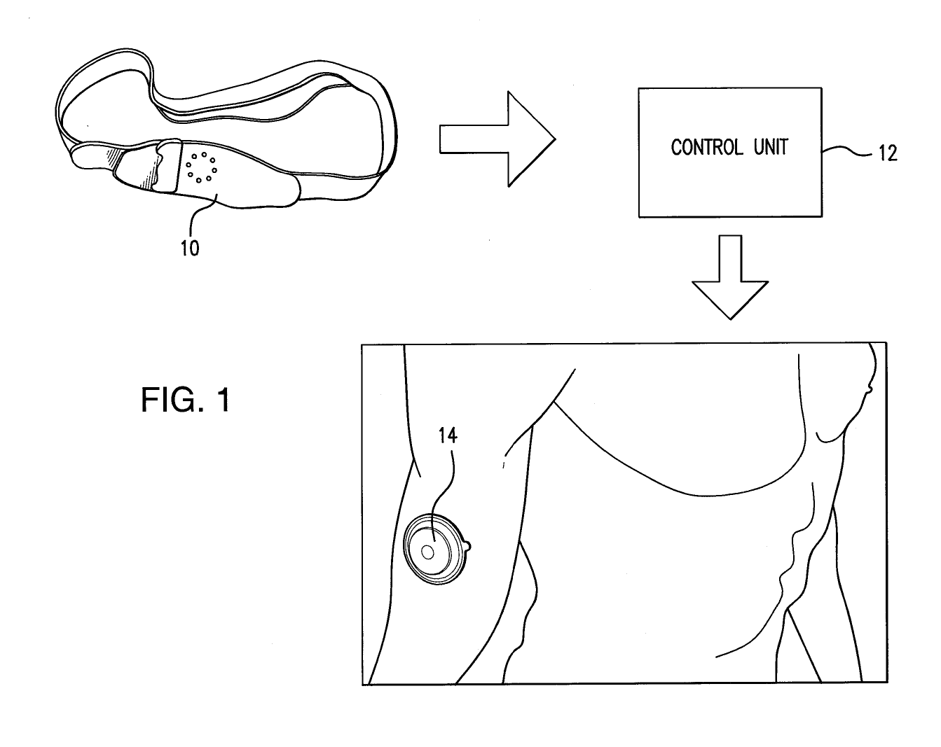

FIG. 1 shows a preferred embodiment of the system of the present invention comprising an electro-physical sensor 10, a control unit 12 and a drug-delivery device 14.

In the embodiment shown in FIG. 1, the sensor 10 is a wireless EEG-type sensor placed on the patient's head and connected via a wireless connection to a polysomnography control unit 12. Control units plus sensors of this type are used to monitor sleep-states; an example commercial model being the ZEO SLEEP MONITOR from Zeo, Inc., Newton, Mass., USA. Alternatively, actigraphy can be used for assessing sleep-state as it is increasingly being employed in sleep clinics to replace full polysomnography. An actigraph-type sensor is typically worn on the wrist and works by analyzing movement; an example commercial model being the ACTISLEEP SLEEP MONITOR from ActiGraph, Inc., Pensacola, Fla., USA. Heart rate sensors can also be used for this purpose. In a preferred embodiment when GH is to be administered, the control unit may be activated by an On/Off switch by the patient (for example when the patient decides to fall asleep) and the hormone will be released at the anticipated time and pattern that mimics the natural GH release profile appropriately selected for the patient based on the patient's age and gender.

The drug-delivery device 14 component of the system is preferably a patch-pump device worn on the body of the patient and able to communicate with the control unit 12, preferably wirelessly, in a manner that would be understood by a person of ordinary skill in the art. Such patch-pump devices typically contain the drug to be delivered and deliver this drug at a flow-rate determined by electronic control; such devices are commercially available from Insulet Corp., Bedford, Mass., USA, and are being developed by a number of further companies including SteadyMed Ltd., Tel-Aviv, Israel.

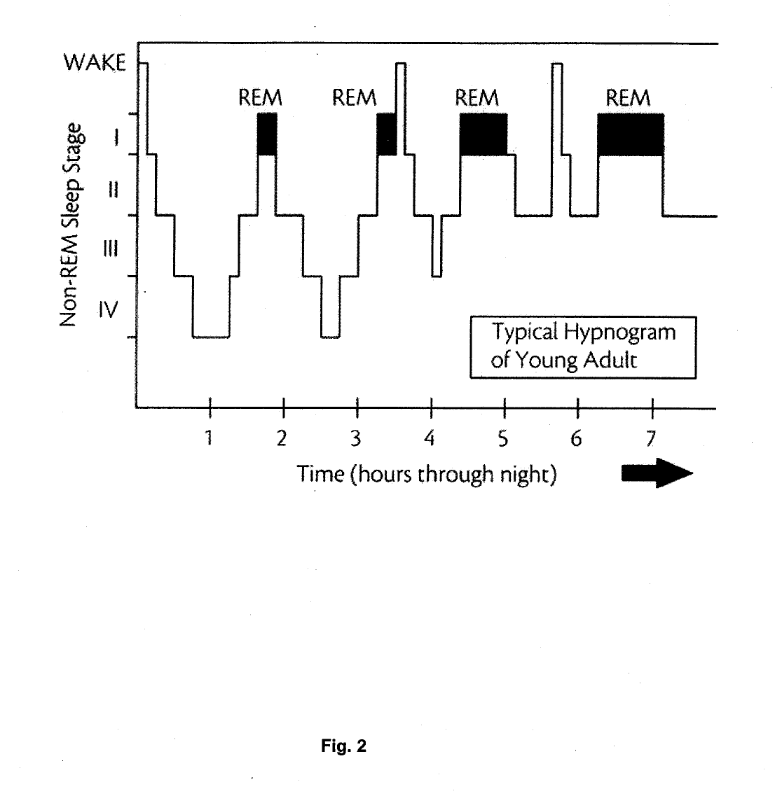

Sleep is typically divided into a number of sleep-states, of which an example trace is shown in FIG. 2. According to the present invention, these sleep-states and/or the transitions between them can for example serve as the trigger for providing a large pulse of bolus of the drug. In a preferred embodiment for patients suffering from GHD, the onset of each deep-sleep stage will trigger a large pulse of GH to be infused by the drug-delivery device 14. This bolus-triggered regimen may be further enhanced by a continuous delivery regimen throughout the sleep state and another bolus each time that sleep state appears and/or continues for the duration of the night or even the entire day. The system of the present invention implements this method of treatment in the following manner: (a) the sensor 10 sends signals to the control unit 12, said signals being reflective of the physical or physiological data that are being measured, (b) the control unit 12 analyses these signals on an ongoing basis and at a certain point in time determines that the sleep-state condition or transition being looked for has occurred, and (c) said control unit 12 then instructs the drug-delivery device to deliver a pre-determined bolus or pulse to the patient. The function relating the sleep-state (including sleep-state transition) to the dose and flow-rate required is set within the software of the controller and/or other software components of the system. Clearly a number of such functions or algorithms can be defined in a manner within the capabilities of one skilled in the art; for example it can be defined that a dose of GH will be delivered (a) every time that there is an entry to a deep-sleep state, (b) only on the first such entry during the night, or (c) using different dose sizes or delivery profiles for each successive entry to a deep-sleep state, etc.

Advantageously, the system and method of the present invention deliver the required hormone at the required dose and timing in terms of the natural release pattern of the GH in the body, thereby minimizing any overdosing of the hormone drug or desensitization due to prolonged exposure. FIG. 3 shows the natural release pattern of growth hormone in adult human males and females. The pattern of natural growth hormone release in children is shown in FIG. 4. This is important as GH can cause a number of medical adverse side effects, including joint swelling, joint pain, carpal tunnel syndrome, and an increased risk of diabetes. This method also increases the efficacy of the therapeutic treatment.

In one embodiment, in the event that no entry to a deep-sleep state has been detected within a preset number of minutes of operation (for example 50-100 minutes), the drug-delivery device will start to deliver the hormone without waiting further. The regimen of delivery in this case will be one that mimics the natural release cycle. This regime can be selected without sleep sensors.

While the invention has been shown herein in what is presently conceived to be the most practical and preferred embodiment thereof, it will be apparent to those of ordinary skill in the art that many modifications may be made thereof within the scope of the invention, which scope is to be accorded the broadest interpretation so as to encompass all equivalent structures and devices.

1. A system for delivering a drug to a patient, said system comprising at least one electro-physical sensor, a control unit and a drug-delivery device adapted to contain at least one drug to be delivered to such patient, said sensor and said control unit being capable of determining a physiological state in a patient wearing said sensor, and said control unit being capable of issuing instructions to said drug-delivery device to deliver the drug to said patient at a flow-rate which is a function of the physiological state determined.

2. The system of claim 1, wherein the drug comprises a hormone.

3. The system of claim 2, wherein said flow-rate follows a profile pattern of hormone release.

4. The system of claim 2, wherein said drug-delivery device delivers missing quantities of hormone at appropriate times according to said patient's body biological clock.

5. The system of claim 1, wherein the physiological state is a sleep-state.

6. The system of claim 1, wherein said control unit comprises a polysomnography controller and said sensor comprises an EEG-type sensor.

7. The system of claim 1, wherein said control unit and said sensor comprise an actigraph or heart rate sensor.

8. The system of claim 2, wherein said control unit comprises a 24 hour clock synchronized with said patient's body biological clock and sleep time and is capable of issuing instructions to said drug delivery device for delivering appropriate amounts of the hormone at appropriate times in order to mimic naturally released hormone amounts over time.

9. The system of claim 2, wherein said control unit is activated with sleep time and issues instructions when slow-wave sleep is expected according to a natural pattern and level of hormone release.

10. The system of claim 1, wherein a dose of said drug is delivered to said patient every time there is an entry to a deep-sleep state.

11. The system of claim 1, wherein a dose of said drug is delivered to said patient on a first entry to a deep-sleep state.

12. The system of claim 1, wherein a different dosage amount of said drug is delivered to said patient upon an entry to a deep-sleep state.

13. The system of claim 1, wherein said drug is delivered to said patient in a different delivery profile upon an entry to a deep-sleep state.

14. The system of claim 1, wherein said system is configured to deliver said drug to said patient within a preset number of minutes of operation.

15. The system of claim 14, wherein said preset number of minutes of operation is about 50 to about 120 minutes.

16. The system of claim 14, wherein said drug is delivered to said patient when no entry to a deep-sleep state is detected.

17. The system of claim 14, wherein said drug is a hormone and is delivered to said patient in a regimen that mimics a natural release cycle of said hormone.

18. The system of claim 14, wherein said sensor is not a sleep sensor.

19. The system of claim 1, wherein said drug-delivery device comprises a patch-pump adhered to the patient's body.

20. The system of claim 1, wherein said sensor is wirelessly connected to said control unit.

21. The system of claim 1, wherein said sensor comprises said control unit.

22. The system of claim 1, wherein said drug-delivery device is wirelessly connected to said control unit.

23. The system of claim 1, wherein said drug-delivery device comprises said control unit.

24. The system of claim 2, wherein the hormone comprises growth hormone or gonadotropin-releasing hormone.

25. The system of claim 8 wherein the drug delivery device can be activated by an On/Off switch by the patient.

26. The system of claim 25, wherein said hormone is released at appropriate times and in a pattern that mimics a natural GH release profile.

27. A method of delivering a drug to a patient in need of such drug, said method comprising:
(a) determining a physiological state in such patient by means of an electro-physical sensor in communication with a control unit;
(b) using said control unit to issue instructions to a drug delivery device; and
(c) administering said drug to said patient by means of said drug delivery device at a flow-rate which is a function of the physiological state determined.

28. The method of claim 27, wherein the drug comprises a hormone.

29. The method of claim 28, wherein the hormone comprises growth hormone or gonadotropin-releasing hormone.
